(19) United States
(12) Patent Application Publication
(10) Pub. No.: US 2011/0117648 A1
(43) Pub. Date: May 19, 2011

(54) SINGLE CELL SURGERY TOOL AND A CELL TRANSFECTION DEVICE UTILIZING THE PHOTOTHERMAL PROPERTIES OF THIN FILMS AND/OR METAL NANOPARTICLES

(75) Inventors: Pei-Yu Chiou, Los Angeles, CA (US); Ting-Hsiang Wu, Los Angeles, CA (US); Michael A. Teitell, Tarzana, CA (US); Sheraz Kalim Butt, South San Francisco, CA (US)

(73) Assignee: THE REGENTS OF THE UNIVERSITY OF CALIFORNIA, OAKLAND (CA)

(21) Appl. No.: 12/667,594

(22) PCT Filed: Jul. 25, 2008
(86) PCT No.: PCT/US08/09090

§ 371 Date: Jan. 26, 2011

Related U.S. Application Data
(60) Provisional application No. 60/952,183, filed on Jul. 26, 2007.

(30) Foreign Application Priority Data
Jul. 18, 2008 (US) 61/082028

(51) Int. Cl. C12N 5/071; C12M 1/02; C12M 1/26; A61M 25/00; B82Y 5/00

(52) U.S. Cl. 435/375; 435/283.1; 435/309.1; 427/2.28; 977/773; 977/762; 977/774; 977/810; 977/838; 977/902; 977/901

(57) ABSTRACT

This invention provides novel tools for surgery on single cells. In certain embodiments the tools comprise a microcapillary having at and/or near the tip a metal coating or a plurality of nanoparticles that can be heated by application of electromagnetic energy. In certain embodiments substrates are provided that facilitate the introduction of agents into cells. The substrates typically comprise a surface bearing a film or particles or nanoparticles that can be heated by application of electromagnetic energy.

### CROSS-REFERENCE TO RELATED APPLICATIONS

This application claims benefit of and priority to U.S. Ser. No. 61/082,028, filed Jul. 18, 2008 and to U.S. Ser. No. 60/952,183, filed Jul. 26, 2007, both of which are incorporated herein by reference in their entirety for all purposes.

### STATEMENT AS TO RIGHTS TO INVENTIONS MADE UNDER FEDERALLY SPONSORED RESEARCH AND DEVELOPMENT

[Not Applicable]

### FIELD OF THE INVENTION

This invention pertains to the fields of microsurgery and cell transfection. In particular, this invention provides a single cell surgery tool and a cell transfection device utilizing the photothermal properties of thin films and/or metal nanoparticles.

### BACKGROUND OF THE INVENTION

Single-cell microinjection is a powerful and versatile technique for introducing exogenous material into cells and for extracting and transferring cellular components between cells, or for the introduction of components not normally found within cells, such as probes, detectors, or genetically engineered organelles or genes. Conventional glass micropipette techniques, such as those used to generate transgenic mice, introduce enormous mechanical and biochemical stresses on the cells and yield low rates of success, particularly for mechanically fragile cells. Current laser-induced cell ablation procedures eliminate this mechanical stress but require highly focused light and the damage volume is diffraction-limited.

### SUMMARY OF THE INVENTION

In certain embodiments the present invention provides a cell surgery tool that can achieve highly local penetration of a cell with significantly reduced damage or stress to the cell. In certain embodiments a cell microsurgery tool is provided where the tool comprises a microcapillary having at and/or near the tip a metal coating or a plurality of nanoparticles that can be heated by application of electromagnetic energy.

In certain embodiments a cell microsurgery tool is provided. Typically, the tool comprises a microcapillary having at and/or near the tip a metal coating or a plurality of nanoparticles that can be heated by application of electromagnetic energy. In certain embodiments the microcapillary comprises a hollow bore. In certain embodiments the tip of the microcapillary ranges in diameter from about 0.01 μm, 0.05 μm, 0.1 μm, or 0.5 μm to about 1 μm, 3 μm, 5 μm, 8 μm, or 10 μm. In certain embodiments the micropipette has an OD ranging from about 0.5 to about 2 μm or 3 μm. In various embodiments the nanoparticles range in size from about 50 nm to about 500 nm. In various embodiments the nanoparticles range in size from about 10 nm to about 500 nm. In various embodiments the nanoparticles are selected from the group consisting of a nanobead, nanowire, a nanotube, a nanodot, a nanocone, and a quantum dot. In various embodiments the metal coating or nanoparticles comprise a noble metal, a noble metal alloy, a noble metal nitride, and/or a noble metal oxide. In various embodiments the metal coating or nanoparticles comprise a transition metal, a transition metal alloy, a transition metal nitride, and/or a transition metal oxide. In various embodiments the metal coating or nanoparticles comprises a magnetic, paramagnetic, or superparamagnetic material. In various embodiments the microcapillary comprises a material selected from the group consisting of glass, a mineral, a ceramic, and a plastic. In certain embodiments the microcapillary comprises a glass microcapillary having nanoparticles near the tip. In certain embodiments the microcapillary comprises a glass microcapillary having gold nanoparticles near the tip. In certain embodiments the microcapillary comprises a glass microcapillary where the nanoparticles are predominantly located within 100 μm of the tip of the microcapillary.

Also provided is a method of performing micromanipulations on a cell. The methods typically involves contacting the cell with a microsurgery tool as described herein; and applying electromagnetic energy to the tool whereby the temperature of the metal coating or metal nanoparticles is increased thereby facilitating penetration of the tool into or through the membrane of the cell. In certain embodiments the applying electromagnetic energy comprises applying light to heat the metal coating or the nanoparticles. In certain embodiments the applying electromagnetic energy comprises applying a laser beam to heat the metal coating or the nanoparticles. While laser heating is generally preferred, other electromagnetic sources are contemplated. Accordingly, in certain embodiments the applying electromagnetic energy comprises applying a magnetic field to heat the metal coating or the nanoparticles. In certain embodiments the applying electromagnetic energy comprises applying an electric field to heat the metal coating or the nanoparticles. In various embodiments the temperature of the metal coating or metal nanoparticles is increased at least 100, 150, 200, 250, 300, or 350 degrees Celsius above-ambient. In certain embodiments the method further comprises injecting a material into the cell through the microcapillary tube. In certain embodiments the method further comprises removing a material from the cell through the microcapillary tube. In certain embodiments the microcapillary comprises a hollow bore. In certain embodiments the tip of the microcapillary ranges in diameter from about 0.1 μm to about 5 μm. In certain embodiments the nanoparticles range in size from about 5 nm to about 500 nm and/or from about 10 nm to about 400 nm. In certain embodiments the nanoparticles are selected from the group consisting of a nanowire, a nanotube, a nanodot, a nanocone, and a quantum dot. In various embodiments the metal coating or nanoparticles comprise a noble metal, a noble metal alloy, a noble metal nitride, and a noble metal oxide. In various embodiments the metal coating or nanoparticles comprise a transition metal, a transition metal alloy, a transition metal nitride, and a transition metal oxide. In various embodiments the metal coating or nanoparticles comprise a magnetic, paramagnetic, or superparamagnetic material. In certain embodiments the microcapillary comprises a material selected from the group consisting of glass, a mineral (e.g., quartz), a ceramic, and a plastic (e.g., DELRIN®, TEFLON®, etc.). In certain embodiments the microcapillary comprises a glass microcapillary having nanoparticles near the tip. In certain embodiments the microcapillary comprises a glass microcapillary having gold nanoparticles near the tip. In certain embodiments the microcapillary comprises a glass microcapillary where the nanoparticles are predominantly located within 100 μm of the tip of the microcapillary.

Also provided is a system for performing microsurgery on a cell, the system comprising a microsurgery tool as described herein, and a micromanipulator (micropositioner) for positioning the microsurgery tool. In certain embodiments the system further comprises a microscope for visualizing a cell manipulated by the microsurgery tool. In certain embodiments the system further comprises a pump for delivering or removing a molecule, organelle, or fluid using the microsurgery tool. In certain embodiments the system further comprises an electromagnetic energy source for exciting the metal nanoparticles and/or thin film on the microsurgery tool. In various embodiments the electromagnetic energy source is selected from the group consisting of a magnetic field generator, a laser, an RF field generator, and the like.

In various embodiments this invention provides methods of preparing a tool for microsurgery on a cell. The methods typically involve attaching to a microcapillary tube a plurality of nanoparticles at or near the tip of the microcapillary tube thereby providing a device that can be locally heated by application of electromagnetic energy to the nanoparticles. In certain embodiments the attaching comprises adsorbing the nanoparticles to the microcapillary. In certain embodiments the attaching comprises fabricating the nanoparticles in situ on the microcapillary. In certain embodiments the attaching comprises chemically coupling the nanoparticles to the microcapillary.

In certain embodiments the single-cell surgery tools of this invention include atomic force measurement (AFM) tips. For example, a nanoparticle can be integrated with an AFM tip for cell surgery applications. This nanoparticle integrated AFM tip can cut any desire shape on a cell membrane by scanning the tip and laser pulsing it.

In certain other embodiments the tools of this invention expressly exclude atomic force measurement (AFM) tips.

Also provided are methods for delivering an agent into a cell. The methods typically involve providing cells on, or adjacent to, a substrate (e.g., a transfection substrate), where said substrate comprises a thin film (e.g., a metallic thin film) capable of being heated by an energy source and/or particles or nanoparticles (e.g., metal nanoparticles) capable of being heated by an energy source (e.g., a laser); contacting the cells with the agent to be delivered; and exposing a region of the substrate with electromagnetic radiation thereby inducing heating of said thin film and/or particles where said heating forms bubbles that introduce openings in the membrane of cells in the heated region resulting in the delivery of said agent into those cells. In certain embodiments the exposing comprises exposing a region of the substrate to a laser. In certain embodiments the substrate comprises nanoparticles and the nanoparticles range in size from about 5 nm to about 500 nm. Suitable nanoparticles include for example, any of the nanoparticles described above. In certain embodiments the nanoparticles include, but are not limited to nanowire, a nanotube, a nanodot, a nanocone, and a quantum dot. In certain embodiments the metal coating or nanoparticles comprise a material selected from the group consisting of carbon, a metal, a metal alloy, a metal nitride, and a metal oxide. In certain embodiments the coating or nanoparticles comprise a material selected from the group consisting of a noble metal, a noble metal alloy, a noble metal nitride, and a noble metal oxide. In certain embodiments the coating or nanoparticles comprise a material selected from the group consisting of a transition metal, a transition metal alloy, a transition metal nitride, and a transition metal oxide. In certain embodiments the coating or nanoparticles comprise a material selected from the group consisting of gold, TiN, TiCn, and TiAlN. In certain embodiments the coating or nanoparticles comprise a magnetic, paramagnetic, or superparamagnetic material. In certain embodiments the substrate comprises a material selected from the group consisting of glass, a mineral, a ceramic, and a plastic. The substrate can optionally comprise a wall and/or floor of a well in a microtiter plate, a microscope slide, a cell culture vessel, and the like. In certain embodiments the agent is a nucleic acid, a chromosome, a cell nucleus, a protein, a label, an organelle, a small organic molecule, and the like. In certain embodiments the cells are mammalian cells.

Also provided are devices for delivering an agent into a cell. In various embodiments the devices comprise a vessel comprising a surface bearing nanoparticles or a thin film of a material that heats up when contacted with electromagnetic radiation. In certain embodiments the device further comprises cells on, adjacent to, or adhered to said surface. In certain embodiments the surface can optionally comprise a wall and/or floor of a well in a microtiter plate, a microscope slide, a cell culture vessel, and the like. In certain embodiments the surface comprises a material such as a glass, a plastic, and a mineral.

In various embodiments systems are provided for selectively delivering an agent into a cell. In certain embodiments the systems typically comprise a vessel comprising a surface bearing nanoparticles or a thin film of a material that heats up when contacted with electromagnetic radiation; and a source of electromagnetic energy capable of heating the nanoparticles or thin film. In certain embodiments the systems further comprises a means of directing the electromagnetic energy to a specific region of the surface. In certain embodiments the means comprises a mask, and/or a focusing system, and/or a positioning system.

Methods are also provided for fabricating a substrate bearing a plurality of nanoparticles. The methods typically involve providing a substrate bearing a metal film; and heating the film whereby said film forms metal nanoparticles adhered to said substrate. In certain embodiments, the substrate comprises a material selected from the group consisting of a metal, a plastic, glass, and quartz. In certain embodiments the substrate comprises a micropipette. In certain embodiments the substrate comprises a wall of a vessel. In certain embodiments the substrate comprises a wall of a cell culture vessel or a microtiter plate. In certain embodiments the film comprises a metal (e.g., a nobel metal, a transition metal, etc.) as described herein for the formation of nanoparticles and/or thin films. In certain embodiments the film comprises a first film that forms nanoparticles and a second adhesion film where said first film is a different material than said second film.

In another embodiments, methods of fabricating a substrate bearing a plurality of nanoparticles, involve providing a substrate; and bombarding the substrate with nanoparticles whereby said nanoparticles adhere to said substrate. In certain embodiments the substrate comprises a material selected from the group consisting of plastic, glass, and quartz. In certain embodiments the substrate comprises a micropipette. In certain embodiments the substrate comprises a wall of a vessel. In certain embodiments the substrate comprises a wall of a cell culture vessel or a microtiter plate. In certain embodiments the nanoparticle(s) can comprise any of the nanoparticles described herein.

### DEFINITIONS

The term “nanoparticles”, as used herein refers to a particle having at least one dimension having an average size equal to or smaller than about 800 nm or 700 nm or 600 nm, or 500 nm, preferably equal to or smaller than about 200 nm or 150 nm, or 100 nm, more preferably equal to or smaller than about 50 or 20 nm, or having a crystallite size of about 10 nm or less, as measured from electron microscope images and/or diffraction peak half widths of standard 2-theta x-ray diffraction scans. In certain embodiments, preferably, the first standard deviation of the size distribution is 60% or less, preferably 40% or less, most preferably 15 to 30% of the average particle size.

The phrase “range in size” with respect to nanoparticle size indicates that the nanoparticles are predominantly within the size range. Thus, for example, in certain embodiments, at least 85%, preferably at least 90%, more preferably at least 95%, and most preferably at least 98%, 99%, or even all of the nanoparticles are within the stated size range.

The term “transition metal” refers to typically refers to any element in the d-block of the periodic table, excluding zinc, cadmium and mercury. This corresponds to groups 3 to 12 on the periodic table.

The terms microcapillary tube and micropipette are used interchangeably. A “microcapillary” is a tube that has a tip with a diameter of less than about 50 μm, preferably less than about 25 μm, more preferably less than about 15 μm or 10 μm, and most preferably less than about 5 μm. In certain embodiments the microcapillary has a tip diameter of about 2 μm or less. In certain embodiments the microcapillary can be a solid rod. In certain embodiments the microcapillary can be replaced with a pipette (capillary tube) having a larger tip diameter (e.g., greater than about 200 nm as described herein).

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide. Preferred “peptides”, “polypeptides”, and “proteins” are chains of amino acids whose α carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to peptide mimetics such as amino acids joined by an ether as opposed to an amide bond.

### BRIEF DESCRIPTION OF THE DRAWINGS

FIG. 1 schematically illustrates one embodiment of the cell-surgery tool.

FIG. 2 shows an SEM image of glass micropipette coated with carbon (left panel) and a TEM image of synthesized gold nanoparticles (right panel).

FIG. 3 shows cells before and after laser pulsing. (Top) Glass micropipette (Middle) carbon coated micropipette (Bottom) gold nanoparticles coated micropipette.

FIG. 4 shows TEM images of aspect ratio 3.2 (A: 24 hr) and 5.8 (C: 72 hr).

FIG. 5 illustrates the fabrication of a transfection substrate using a biolistic injector (left panel) to bombarding gold particles directly onto a plastic substrate. The middle panel a typical particle on the substrate, while the right panel illustrates the particle density on the substrate.

FIG. 6, panels A-D, shows a method of forming nanoparticles on a surface by heating a thin film (e.g., using a pulsed laser) to form annealed particles. In this example, a 30 nm gold film with 2 nm titanium adhesion layer was heated with a pulsed laser (532 nm, 6 ns) at 113.2 mJ/cm² for 100 pulses. Particle size ranged from 0.1 to 0.7 μm (see, e.g., panel C). Particle density was about 0.65 particle/μm²=65 particle per cell (assuming cell area˜10 μm×10 μm).

FIG. 7 illustrates a schematic of one embodiment of a device capable of light-patterned molecular delivery using a gold particle coated substrate.

FIG. 8 illustrates a schematic of an experimental setup for light-patterned molecular delivery and a time-resolved imaging system used to capture the cavitation bubble dynamics.

FIG. 9 shows bubbles induced by pulsed laser irradiation on the gold particles. (a) Before the laser pulse (b) 78 ns after the laser pulse. Bar=50 μm.

FIG. 10 shows light-patterned fluorescence dye uptake in HEK293T cells using a shadow mask. (a) Bright field (b) Fluorescent image (mask covered the side to the right of the dash line.

FIG. 11 shows preliminary photothermal pipette tests on 293T cells. Before (left column) and after (right column) laser pulsing. Pipettes were coated with Au/Pd thin films of different thickness. Film thickness=5 nm (panel a) versus 13 nm (panel b). The laser fluence used in both experiments was 88.3 mJ/cm².

FIG. 12, panels a-c, show microinjection of GFP-encoding plasmids into adherent 293T cells using gold thin-film coated pipette. Panel a: Microinjection procedure, Panel b: A fluorescent image of cells 24 hours after injection, showing cells viable and expressing GFP; Panel c: An image overlaying the phase contrast and the fluorescence images.

FIG. 13 shows a schematic of non-adherent cell microinjection using plasmonic photothermal pipette combined with optical tweezers (top panel) and an image showing a Nalm-6 cell trapped by optical tweezers while in contact with the photothermal pipette tip (bottom panel).

### DETAILED DESCRIPTION

In certain embodiments this invention pertains to a new tool useful for “surgical” procedures on single cells. In various embodiments the tool is designed to perform microinjection, microextraction, and/or intracellular manipulations with minimal cell damage. It can be used with any cell bounded by a lipid bilayer (e.g., a vertebrate, more preferably a mammalian cell type). In certain embodiments the device can be utilized with cells comprising a cell wall (e.g., plant cells).

In certain embodiments the device achieves precise and controllable nanoscale cell modification, by laser excitation of specific-aspect ratio metal particles/nanoparticles and/or a metal film (e.g., nanocoating) coated on a pipette (microcapillary). This tool facilitates operative procedures on small and mechanically fragile cells with a high rate of success.

Single-cell microinjection is a powerful and versatile technique for introducing exogenous material into cells, for extracting and transferring cellular components between cells, and/or for the introduction of components not normally found within cells, such as probes, detectors, or genetically engineered organelles, genes, proteins and the like. Conventional glass micropipette techniques, such as those used to generate transgenic mice, introduce enormous mechanical and biochemical stresses on the cells and yield low rates of success, particularly for mechanically fragile cells.

Current laser-induced cell ablation procedures eliminate this mechanical stress but require highly focused light and the damage volume is diffraction-limited. In contrast, the cell surgery tool describe herein can achieve manipulations (e.g., ablation) at a nanometer size scale at or near the tip of a capillary micropipette by local heating of particles/nanoparticles and/or a thin film at or near the pipette tip. The particles/nanoparticles and/or thin film are heated using electromagnetic radiation (e.g., a magnetic field, an electric field, an RF field, broad or focused laser pulses, and the like).

In various embodiments the cell surgery utilizes the photothermal effect of metal particles/nanoparticles and/or a thin metal film. For example, by controlling the geometry and/or composition of the particles and/or the thickness or composition of the film, the material can be “tuned” so that electromagnetic radiation (e.g., laser energy) is strongly absorbed by particles and/or thin film, but not by nearby cells or cellular contents, thereby avoiding cell or genetic damage caused by traditional laser-based methods of manipulating cells. Current laser cell techniques rely on strong absorption of the laser power by cellular contents to create ablation or cavitation effects. Such processes can damage cells and can cause undesired breakdown of cellular constituents or chemical effects that may affect the biology of the cells being manipulated.

In contrast, utilizing the microsurgical tools described herein, the laser, or other source of electromagnetic energy, is used to heat particles/nanoparticles and/or a thin film localized at the cell membrane by being bound to microcapillary pipettes. Thus, the energy source (e.g., laser) does not substantially damage the cells being manipulated.

Depending on the selection of materials, the nanoparticles and/or thin film can be excited (heated) by application of essentially any electromagnetic radiation. Thus, in various embodiments, heating of the nanoparticles and/or thin film(s) is accomplished by application of a magnetic field, and/or an electric field, and/or an RF field, and/or light (e.g., a laser).

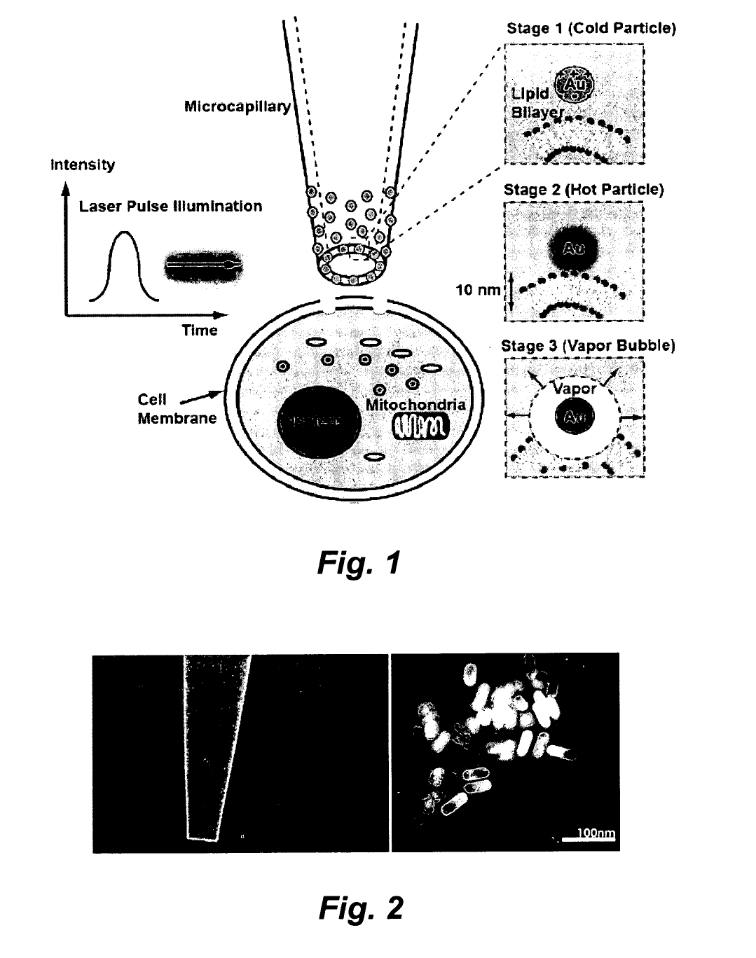

For example, noble metal particles, nanoparticles, and thin films strongly absorb electromagnetic waves with frequencies close to the surface plasmon frequency, usually in the visible and near-IR range. Particles, nanoparticles, and thin films rapidly heat up, due to the absorbed energy, to generate a superheating phenomenon with evaporation of the surrounding medium. In certain embodiments of the cell surgery tool, individual or multiple nanoparticles are coated onto the tip of a micropipette, e.g., as shown in FIG. 1. Upon laser pulse excitation, nanometer diameter vapor bubbles are created around the nanoparticles. When brought nearby or in contact with the surface of a cell, this process generates controlled, precisely-sized holes in the cell membrane (and/or cell wall). This way the micropipette can penetrate the cell membrane with ease without inducing mechanical and biochemical damage associated with current microinjection and extraction techniques. The cavitation or “hole punching” process is finished within a few nanoseconds. As a result, the rest of the membrane does not have time to respond and remains mechanically undisturbed. Once the pipette is in place in the cell, the “membrane hole” can be kept open for manipulations with devices, such as fiber optic devices, threaded through the hollow bore of the pipette. In certain embodiments similar effects can be obtained using thin metal film deposited at and/or near the tip of the microcapillary.

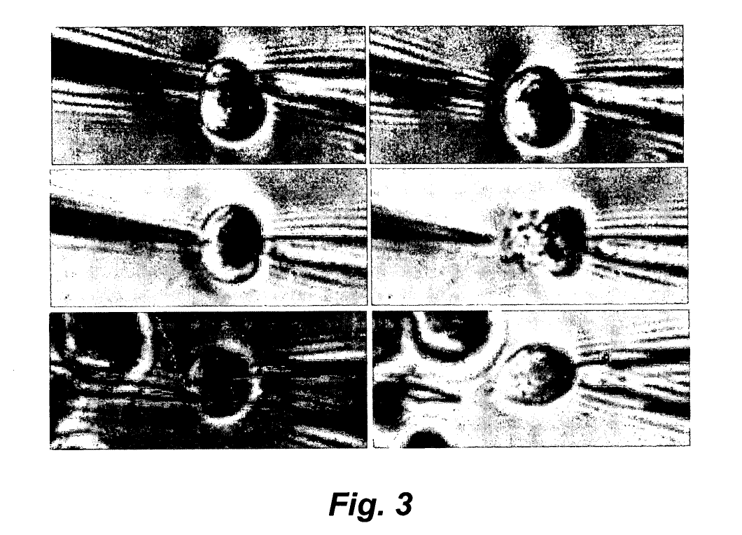

The cell surgery tool can be used for performing single-cell microinjection, and/or extraction, and/or intracellular manipulation. Hole punching on adherent cell membranes has been demonstrated, and this is easily extended to cells in suspension, to cells immobilized using optical tweezers, and/or to cells that routinely grow in clusters, colonies, or clumps with the assistance of a standard suction-based holding pipette. Illustrative experimental results are shown in FIG. 3.

The cell surgery tool can provide precise intracellular access through specifically-sized membrane holes that remain open for periods determined by the operator, or that close rapidly as needed, with minimal to no cell damage for live-cell manipulations. The device can be used to increase the efficiency and success rate for performing pronuclear DNA microinjection, embryonic stem cell transfer into blastocysts, somatic cell nucleus transfer, repair or replacement of other intracellular organelles, for the introduction of non-cellular materials, such as probes, and the like.

In various embodiments an injection micropipette is fabricated using, for example, a commercial pipette puller. Nanoparticles (e.g., noble metal nanoparticles such as gold, silver, tantalum, platinum, palladium, or rhodium nanoparticles) of particular size and/or aspect ratio are coated onto the tip of the micropipette. The micropipette is positioned next to the targeted cell membrane by micromanipulators and/or automated (e.g., piezo-driven) stages under a microscope (e.g., an inverted microscope). By pulsing a laser (or other energy source) on the tip of the micropipette, the pipette eases through the membrane without causing significant cell deformation. Once the pipette is inside the cell, subsequent injection or extraction of molecules and cellular components can be performed, as can live cell intracellular manipulations.

In another embodiment, methods, devices, and systems are provided for the delivery of agents (e.g., nucleic acids, proteins, organic molecules, organelles, antibodies or other ligands, etc.) into live cells. Typically the devices comprise a substrate bearing particles (e.g., nanoparticles) and/or a thin film (e.g., as described above). Cells are seeded on this substrate and, optionally grown until a confluent culture forms. In certain embodiments a pulsed laser (or other electromagnetic energy source) irradiates a shadow mask and the corresponding illumination pattern is imaged onto the substrate. In the area exposed to the energy source (e.g., pulsed laser) the particles and/or thin film is heated to high temperatures due to the absorbed energy. Typically, within a few nanoseconds, the heat is dissipated into the liquid medium layer surrounding the particles and/or thin film, thereby generating vapor bubbles. The rapid expansion and subsequent collapse of the vapor bubbles gives rise to transient fluid flows that induce strong shear stress on the nearby cell(s) causing localized pore formation in the cell membrane (and/or cell wall). As a result, membrane-impermeable molecules can be carried into the cell by fluid flows or thermal diffusion. Since the cavitation bubbles) only form where the particle(s) and/or thin film is exposed to the energy source, irradiation patterns can be selected/designed that induce molecular uptake in specified areas of the cell culture and/or at specified times. In this way high-throughput, spatially-targeted and/or temporally-targeted molecular delivery is made possible by controlling the particle size, material, and/or density on the substrate and/or film material and/or thickness on the substrate, the energy source timing, intensity and frequency, and the irradiation pattern(s).

It will be appreciated that any means of locally/selectively illuminating the substrate can be used. Thus, for example, in certain embodiments, local illumination of a particular region of the substrate can be accomplished by using a mask (shadow mask). In certain embodimenst, local illumination can be achieved simply by focusing the illuminating energy source (e.g., laser) to a particular region using a lens and/or mirror system. In certain embodiments the energy source can be focused at a fixed region and the substrate moved (e.g., using a movable stage or other manipulator) to achieve local illumination of particular regions.

In certain embodiments the laser pulses can be shaped by not only the static shadow masks as demonstrated in the examples, but also by dynamic masks using a spatial light modulator such as a TI's DMD microdisplay or LCD display. This provides real-time and interactive control of microinjection into target cells.

These “addressable” delivery devices can be used in a wide variety of contexts. For example, in high throughput systems (different wells or different regions of a single well can have reagents selectively delivered into the target cells simply by administering the agent to the medium and irradiating (e.g., with laser radiation) the region containing the cells into which the agent is to be transported. The first agent can then be washed out, a second agent applied, and a different region irradiated thereby producing cells transfected with different agents at different locations in the culture. This facilitates massively parallel processing of cells permitting the extraordinary control over the timing and spatially addressable delivery of one or more agents.

In certain embodiments the devices described herein can be integrated for example, with other microfluidic devices with pumps and valves (e.g., lab on a chip) for delivery of particular agents to cells.

### Fabrication of Substrates for Addressable Delivery of Agents to Cells and Fabrication of Micropipettes for Single Cell “Surgery”.

In certain embodiments single cell surgery devices are provided comprising a micropipette having at or near the tip particles or nanoparticles and/or thin films that can be heated using a source of electromagnetic energy (e.g., a laser). In various embodiments cell transfection devices are provided comprising a substrate (e.g. a cell culture vessel, a microtiter plate, etc.) comprising nanoparticles and/or thin films that can be heated using a source of electromagnetic energy (e.g., a laser).

In various embodiments the particles and/or nanoparticles or thin film(s) comprising the various devices described herein comprise a single material. In other embodiments, the particles and/or nanoparticles or thin film(s) comprise two or more materials. Thus for example, particles or nanoparticles comprising the devices can be populations having two, three, four, five, or more different types of particle (e.g., particle size, and/or shape, and/or material). Similarly, the thin films comprising the devices can comprise multiple films (e.g., as multiple layers, or different films at different locations on the micropipette or substrate). For example, the devices can be modified by coating another layer of metal, or dielectric materials (e.g., silicon oxide, silicon nitride, etc.) on top of the metal thin film to control the heat dissipation pathways and to control the microbubble explosion patterns which effects the amount of area on the cell that is damaged by the substrate heating or by the pipette tip.

Micropipette and Substrate Fabrication.

The micropipettes and “transfection substrates” are fabricated using methods well known to those of skill in the art. For example, in various embodiments an injection micropipette is fabricated using, for example, a commercial pipette puller

The micropipette can be fabricated from any material that can be pulled, etched, or otherwise fabricated to the desired dimension(s) while providing the requisite stiffness and heat resistance to permit heating and cell penetration. In addition, the material is preferably not toxic to the cell. In certain embodiments the microcapillary comprises a material such as glass, a mineral (e.g., quartz), a ceramic, a plastic (e.g., DELRIN®, TEFLON®, etc.), a metal, a semiconductor, and the like.

When the micropipettes is fabricated, the cross-sectional shape of the pipette is typically circular. However, this does not mean that only circular pipettes can be used for the cell surgery tool. Micropipettes having other cross-sections can be fabricated. For example, pipette tips with any desired patterns, circular, rectangular, or triangular can be fabricated on a glass or silicon wafer first and then transferred and assembled with an injection pipette.

In addition, a number of pre-pulled micropipettes are commercially available (see, e.g., World Precision Instruments, Hertfordshire, England).

Pipette diameter an important parameter for the single-cell surgery instrument. One advantage of the single-cell surgery instrument described herein is that it allows opening a gm size hole in a cell membrane with less collateral damage to the cell. This allows the delivery large size DNAs or other materials into a cell without killing it. To reduce collateral damage, conventional micropipette techniques typically require the outer tip diameter of a glass pipette to be smaller than 200 nm to facilitate the penetration across the flexible cell membrane of small mammalian cells. This restriction greatly limits the size of particles that can be delivered through conventional micropipette techniques. Materials such as chromosomes, nuclei, organelles, or other extracellular materials have great research and commercial value, but often have a size larger than the physical size of the pipette opening and cannot be introduced into cells through conventional techniques. Our laser cell surgery pipette provides a unique solution to this problem which has the potential to revolutionize the entire micropipette industry. In general, the methods and devices of this invention are effective to introduce large DNA fragments (e.g., BAC-sized or larger DNA), nuclei, organelles, and other large moieties into cells more efficiently with less cell damage than other techniques. Accordingly, in certain embodiments, the micropipette comprising the single cell surgery tool described herein has a tip diameter greater than 200 nm, in certain embodiments greater than about 300 nm, greater than about 400 nm, greater than about 500 nm, greater than about 600 nm, greater than about 700 nm, greater than about 800 nm, or greater than about 900 nm or 1 μm.

Similarly the substrate(s) comprising the “transfection substrate” can be fabricated from any convenient material that is preferably not toxic to the cell(s), that can carry the particle, nanoparticle, or thin film coating, and that can tolerate the local heating produced by application of electromagnetic energy to the particles, nanoparticles, and/or thin film(s). Suitable materials include, but are not limited to glass, a mineral (e.g., quartz), a ceramic, a plastic (e.g., DELRIN®, TEFLON®, etc.), a metal, a semiconductor, and the like.

In various embodiments, the substrate is a surface of a vessel used for cell screening and/or for cell culture. This can include, for example, vessels for adherent or suspended cell culture. This can also include, microtiter plates (e.g., 96, 384, 864, 1536 well, etc.), microfluidic devices, high density (microarray) substrates, microscope slides or chambers, and the like.

Particle/Nanoparticles/Thin Film Materials

In various embodiments the particles/nanoparticles and/or thin films on the micropipette and/or “transfection substrate” are fabricated from a metal or metal alloy that can be heated by the application of appropriate electromagnetic energy. In various embodiments noble metal, noble metal alloys, and oxides and/or nitrides thereof are contemplated. Depending on size, aspect ratio, film thickness, and/or material, such metals or metal alloys are readily heated using various energy sources (e.g., laser light, electric field, RF field, magnetic field, ultrasonic source, etc.).

While most of the discussion provided herein pertains to metal particles/nanoparticles and/or films, and the examples describe gold particles, the materials heated by the energy source need not be so limited. Essentially any material that absorbs the appropriate energy with resultant heating can be used for the heating material in the methods and devices described herein. Thus, for example, we have demonstrated that black carbon can also induce explosive bubbles near the pipette and kill or penetrate a single cell. Accordingly, in certain embodiments, for example, carbon nanoparticles, including, but not limited to carbon nanotubes can be used in the methods and devices described herein.

One important material useful in the devices and methods described herein is tin nitride (TiN), which is a very hard material with a melting temperature three times higher than gold. When coated on a pipette, 40 cells have been consecutively injected using one single pipette without seeing any pipette damage. This also means that a TiN coated pipette can potentially inject hundreds or thousands of cells without the need to change the pipette. This is a significant improvement as compared to a gold coated pipette, which can be damaged by the strong explosive bubbles and high temperature excitation in one, two, or a few uses.

Other variants of TiN are well known to those of skill in the art. These include, but are not limited to titanium carbon nitride (TiCN) and titanium aluminium nitride (TiAlN), which can be used individually or in alternating layers with TiN or in mixed particle populations with TiN particles. These coatings offer similar or superior enhancements in corrosion resistance and hardness, and different (even tunable) absorption properties.

In addition to laser energy, magnetic, electric fields, and RF fields can also readily be used to heat particles, nanoparticles and/or certain thin films. Thus, for example, U.S. Patent Publication No: 2007/0164250, which is incorporated herein by reference, provides magnetic nanoparticles, that when placed in a magnetic field are selectively heated at a certain frequency of the magnetic field, as a function of their size, composition, or both.

In various embodiments such nanoparticles or coatings comprise magnetic materials (such as the Ferro V magnetic pigment) that transduce energy when exposed to a magnetic field of sufficient intensity. Thus, for example, an alternating magnetic field will induce an alternating current in the particle, producing heat. A variety of magnetic materials can be used. Such materials include, but are not limited to magnetic materials, such as Fe—O₄, Fe₂O₃. Also, in certain embodiments, silver, copper, platinum, palladium and the like can comprise the particles, nanoparticles, and/or thin films used in the devices of this invention. In certain embodiments the particles, nanoparticles, and/or thin films can comprise TiO₂, CeO₂, Ag, CuO, yttrium aluminum garnet (YAG), InO₂, CdS, ZrO₂, or a combination thereof. In another embodiment, any metal oxide, metal alloy, metal carbide, and/or transition metal, may be used in the instant invention. In some embodiments, the particles can be coated, such that the coating does not alter their respective responsiveness to the applied field.

In certain embodiments, particles, nanoparticles, or thin films used in the devices of the present invention can be made of magnetic materials, while in other embodiments, they can be made of or comprise paramagnetic or superparamagnetic materials.

Accordingly, in certain embodiments the particles, nanoparticles and/or thin films can comprise a paramagnetic or superparamagnetic material that can be heated using electron spin resonance absorption (SPM) and/or ferromagnetic resonance. Electron spin resonance (ESR) heating and ferromagnetic resonance (FMR) heating are described in US Patent Publications 2006/0269612 and 2005/0118102, which are incorporated herein by reference. Yttrium-iron garnet Y₃Fe₅O₁₂ and γ-Fe₂O₃ are two well-known materials suitable ESR and/or FMR heating. Different dopants can be added to lower the spin resonance frequencies of these materials various applications. Magnetic garnets and spinels are also chemically inert and indestructible under normal environmental conditions.

Also contemplated are various materials and/or semiconductors comprising materials from Groups II, III, IV, and V of the periodic table.

An illustrative list of potential dilutant ions for the generic {c}₃(a)₂[d]₃O₁₂ and spinel A[B]₂O₄ ferrite compounds are presented in Table 1.

TABLE 1
Illustrative ferrite diluent ions.

| | |
|---|---|
| Garnet {c}₃(a)₂[d]₃O₁₂ | Spinel A[B]₂O₄ |

| {c} | (a) octahedral | A | [B] |
|---|---|---|---|
| dodecahedral | [d] tetrahedral | tetrahedral | octahedral |
| Y³⁺ | Fe³⁺ | Fe³⁺ | Fe³⁺ |
| La³⁺ | Mn²⁺ | Mn²⁺ | Mn²⁺ |
| Gd³⁺ | Ru³⁺ | Ru³⁺ | Ru³⁺ |
| Eu²⁺ | Cu¹⁺ | Cu¹⁺ | Cu¹⁺ |
| Na¹⁺ | V³⁺ [d], Ni²⁺ (a) | V³⁺ | Ni²⁺ |
| K¹⁺ | Cr⁴⁺ [d], Cu³⁺ (a) | Cr⁴⁺ | Cu³⁺ |
| Rb¹⁺ | Mo⁴⁺ [d], Cr³⁺ (a) | Mo⁴⁺ | Cr³⁺ |
| Tl¹⁺ | W⁴⁺ [d], Mo³⁺ (a) | W⁴⁺ | Mo³⁺ |
| Ag¹⁺ | Nb³⁺ [d], W³⁺ (a) | Nb³⁺ | W³⁺ |
| Au¹⁺ | Zn²⁺ | Zn²⁺ | Zn²⁺ |
| Hg¹⁺ | Mg²⁺ | Mg²⁺ | Mg²⁺ |
| Ca²⁺ | Al³⁺ | Al³⁺ | Al³⁺ |
| Sr²⁺ | Ga³⁺ | Ga³⁺ | Ga³⁺ |
| Ba²⁺ | In³⁺ | In³⁺ | In³⁺ |
| Hg²⁺ | Sc³⁺ | Sc³⁺ | Sc³⁺ |
| Pb²⁺ | Ti⁴⁺ | Ti⁴⁺ | Ti⁴⁺ |
| Bi³⁺ | Zr⁴⁺ | Zr⁴⁺ | Zr⁴⁺ |
| In³⁺ | Hf⁴⁺ | Hf⁴⁺ | Hf⁴⁺ |
| Sc³⁺ | Si⁴⁺ | Si⁴⁺ | Si⁴⁺ |
| | Si⁴⁺ | Si⁴⁺ | Si⁴⁺ |
| | Ge⁴⁺ | Ge⁴⁺ | Ge⁴⁺ |
| | Sn⁴⁺ | Sn⁴⁺ | Sn⁴⁺ |
| | V⁵⁺ | V⁵⁺ | V⁵⁺ |
| | Nb⁵⁺ | Nb⁵⁺ | Nb⁵⁺ |
| | Ta⁵⁺ | Ta⁵⁺ | Ta⁵⁺ |
| | P⁵⁺ | P⁵⁺ | P⁵⁺ |
| | As⁵⁺ | As⁵⁺ | As⁵⁺ |
| | Sb⁵⁺ | Sb⁵⁺ | Sb⁵⁺ |

The particle or nanoparticles can take any of a number of possible morphologies and still be suitable for use in the present invention. Thus, for example, this invention contemplates using nanotubes of the following kinds: single walled, double walled, multi walled, with zig-zag chirality, or a mixture of chiralities, twisted, straight, bent, kinked, curled, flattened, and round; ropes of nanotubes, twisted nanotubes, braided nanotubes; small bundles of nanotubes (e.g., in certain embodiments, with a number of tubes less than about ten), medium bundles of nanotubes (e.g., in certain embodiments, with a number of tubes in the hundreds), large bundles of nanotubes (e.g. in certain embodiments, with a number of tubes in the thousands); nanotorii, nanocoils, nanorods, nanowires, nanohorns; empty nanocages, filled nanocages, multifaceted nanocages, empty nanococoons, filled nanococoons, multifaceted nanococoons; thin nanoplatelets, thick nanoplatelets, intercalated nanoplatelets, nanocones, and the like. The various nanoparticles (nanostructures) can assume heterogeneous forms. Such heterogeneous forms include, but are not limited to structures, where one part of the structure has a certain chemical composition, while another part of the structure has a different chemical composition. An example is a multi walled nanotube, where the chemical composition of the different walls can be different from each other. Heterogeneous forms also include different forms of nanostructured material, where more than one of the above listed forms are joined into a larger irregular structure. In addition, in certain embodiments any of the above materials can have cracks, dislocations, branches or other impurities and/or imperfections.

In certain embodiments, the size of the particles or nanoparticles and/or the area and/or thickness of the thin film(s) for use in the present invention can be adjusted or optimized and reflect the choice of the nanoparticles or film material, the nature of the excitation energy, and frequency and/or strength of the excitation energy. In certain embodiments the nanoparticles range in size (e.g., length and/or width and/or diameter) from about 10 to about 500 nm, preferably from about 20 nm to about 200 nm, more preferably from about 20 nm, 30 nm, 40 nm or 50 nm to 100 nm, or 150 nm or 200 nm. In certain embodiments the size of the nanoparticles for use in the present invention ranges from about 4 nm to about 25 nm, in another embodiment, from 8 nm to 5 nm, in another embodiment from 5 nm to 100 nm, in another embodiment, from 10 nm to 800 nm, in another embodiment, from 10 nm to 50 nm, in another embodiment, from 50 nm to 200 nm, and in another embodiment from 150 nm to 500 nm.

In various embodiments, where present, metal films range in thickness from about 1, 2, 5, 10, 50, 100, 150, 200, 300, 400, or 500 nm to about 800 nm, 1 μm, 5 μm, 10 μm, 50 μm, or 100 μm. In certain embodiments the metal films range in thickness from about 2 nm or 5 nm, 10 nm, 20 nm, or 30 nm to about 100 nm, 300 nm, 500 nm, 800 nm or 1 μm. In certain embodiments the metal films range in thickness from 1 nm to 150 nm, preferably from about 5 nm to 100 nm, more preferably from about 5 nm, 10 nm, or 20 nm to about 50 nm, 75 nm, or 100 nm. In certain embodiments the metal films are about 30 nm in thickness.

In various embodiments the coated layer comprising the devices described herein can be a continuous thin film, a thin film broken up into small domains (e.g., 5 nm, 10 nm, 20 nm, 50 nm, 100 nm, 200 nm, 500 nm domains), or can comprise discrete particles or nanoparticles as described herein. The shape of the particles and the thickness of thin films as well as the particle or film composition will affect the absorption spectrum of the material and the energy source and intensity required to produce the desired local heating.

In general, the film thickness and/or particle size effects the size of the bubble(s) produced by local heating and the nature of the microfluidic flow near the bubbles. This determines the shear stress produced and the size of the opening(s) produced in the cell. In general, the larger the particles, the larger the bubbles produced and the more impact produced on the cell. The thickness of thin films has a similar effect as the particle size. The thicker the film, the larger the bubble produced and the larger the hole(s) produced in the cell(s).

Fabrication of Particles/Nanoparticles and Application of Particles and/or Films to Microcapillaries.

Methods of manufacturing particlesor nanoparticles and depositing them on a surface or synthesizing such particles in situ on a surface and methods of depositing thin films on surfaces are well known to those of skill in the art.

For example, thin films can be deposited by any suitable method including but not limited to sputtering deposition, chemical vapor deposition (CVD), molecular beam epitaxy (MBE), plasma-assisted vapor deposition, cathodic arc deposition or Arc-PVD, and electron beam evaporation deposition. In the single cell surgery devices, in various embodiments the film will partially or fully cover the tip of the microcapillary to a distance of up to 100 μm, 50 μm, 25 μm, 10 μm, or 5 μm from the tip, to a distance of up to 1 μm from the tip, more preferably to a distance of up to 800 nm, preferably up to 500 nm, more preferably up to 300, 200, 150, 100, 50, or 25 nm from the tip. Thin films can also be chemically deposited on the microcapillary or cell transfection substrate.

Methods of fabricating particles and nanoparticles are also well known to those of skill in the art. Such methods include, but are not limited to combustion synthesis (e.g., using an oxidizer (e.g., metal salt) and a fuel (e.g., organic compounds) in a redox reaction), evaporation/condensation (EC) generators, spray pyrolysis (e.g., plasma processing and powder spray), liquid phase methods using solution chemistry such as supercritical fluids, chemical reduction, or chemical oxidation, mechanical alloying, template methods (e.g., forming nanoparticles within small voids or areas. Zeolites, pillared clays, nanoporous membranes and inverse micelles), and the like (see, e.g., U.S. Pat. Nos. 7,212,284, 7,204,999, 7,147,712, 7,128,891, 6,972,046, 6,688,494, 5,665,277 which are all incorporated herein by reference, and PCT Patent Application No: WO/2007/024323, which is incorporated herein by reference). The production of nanohorns is described, e.g., by Berber et al. (2000) Physical Review B, 62(4): R2291-2294, while the production of nanofibers is described, for example in U.S. Pat. Nos. 6,706,248, 6,485,858, which are incorporated herein by reference. See also, Fedlheim and Colby (2001) Metal Nanoparticles: Synthesis Characterization & Applications, Marcel Dekker, Inc., N.Y.; Baraton (2002) Synthesis, Functionalization and Surface Treatment of Nanoparticles, American Scientific Publishers; Fendler (1998) Nanoparticles and Nanostructured Films: Preparation, Characterization and Applications, Wiley-VCH, N.Y.; and the like.

In certain embodiments the nanoparticles are synthesized in surfactant systems. Such surfactant-based methods are well known to those of skill in the art. One such approach is illustrated in Example 1.

More generally, in certain embodiments, nanoparticles can be formed on the reduction of metallic salts by organic solvents (e.g., ethanol) to form metal colloids (see, e.g., Hirai et al. (1979) J. Macromol. Sci. Chem., A13: 727; Hirai et al. (1976) Chem. Lett., 905; Toshima and Yonezawa (1992) Makromol. Chem., Macromol. Symp., 59: 281; Wang and Toshima (1997) J. Phys. Chem., 97: 11542, and the like). One illustrative approachy is described by Pastoriza-Santos and Liz-Marzan (2000) Pure Appl. Chem., 72(1-2): 83-90. In their approach, Ag⁺ ions are reduced by N,N-dimethylformamide (DMF) in the presence or absence of a stabilizing agent. The reaction leads to the formation of either thin films of silver nanoparticles electrostatically attached onto surfaces, or stable dispersions of silver nanoparcicles.

In another illustrative approach, magnetite nanoparticle materials can be made by mixing iron salt with alcohol, carboxylic acid and amine in an organic solvent and heating the mixture to 200-360° C. The size of the particles can be controlled either by changing the iron salt to acid/amine ratio or by coating small nanoparticles with more iron oxide. Magnetite nanoparticles in the size ranging from 2 nm to 20 nm with a narrow size distribution can readily be obtained. The method can easily be extended to other iron oxide based nanoparticle materials, including MFe₂O₄ (where M is for example Co, Ni, Cu, Zn, Cr, Ti, Ba, Mg, and the like) nanomaterials, and iron oxide coated nanoparticle materials. The method also leads to the synthesis of iron sulfide based nanoparticle materials by replacing alcohol with thiol in the reaction mixture. The magnetite nanoparticles can be oxidized to γ-Fe₂O₃, or α-Fe₂O₃, or can be reduced to bcc-Fe nanoparticles, while iron oxide based materials can be used to make binary iron based metallic nanoparticles, such as CoFe, NiFe, and FeCoSmx nanoparticles (see, e.g., U.S. Pat. No. 7,128,891, which is incorporated herein by reference).

One method of producing gold nanoparticles involves mixing a gold salt solution with an adsorbent. Gold in the form of complexes is adsorbed onto the surface of the adsorbent. The gold-loaded adsorbent, after being separated from the solution by screening, filtration, settling or other methods, is ashed to form ashes. The ashes contain gold nanoparticles and impurities such as oxides of sodium, potassium and calcium. The impurities can be removed by dissolution using dilute acids. The relatively pure gold nanoparticles are obtained after the impurities are removed. Activated carbon or gold-adsorbing resin can be used as the adsorbent. Silver or platinum group metal nanoparticles can also readily be produced by this method (see, e.g., U.S. Pat. No. 7,060,121, which is incorporated herein by reference.

In still another approach, nanoparticles, can be formed using laser pyrolysis. Conventional laser pyrolysis processes, often called photothermal processes, are well known to those of skill in the art (see, e.g., U.S. Pat. Nos. 5,958,348, 3,941,567. 6,254,928, which are incorporated herein by reference, and the like). In this process, a radiation absorber or other precursor gaseous species absorbs energy (e.g., laser light, which results in the heating of the materials in a reaction zone causing thermally driven chemical reactions between the chemical components in the reaction zone. Typically, laser pyrolysis processes employ a precisely defined hot zone (typically 1000-1500° C.) generated, e.g., by a laser beam passing through a chemical vapor zone, in which gases thermally react to form the desired nanoscale particulate materials. The absence of wall in contact with the hot zone eliminates any contamination.

The materials formed in the pyrolytic reaction leave the hot zone typically driven by gravity or gas flow. The materials are rapidly cooled/quenched thereby forming nanoparticles with a very uniform distribution of sizes and shapes. In typical embodiments, a carbon dioxide (CO₂) laser is used to heat the gas molecules directly by light absorption. Another advantage of using a laser is its narrow spectral width, which allows efficient coupling between the light and the molecular precursor that has exact wavelength of absorption (over 15% of laser power consumed). The technology has been used to produce various nanosize materials from metals, metal carbides, metal nitrides and metal oxides (see, e.g., Haggerty et al. (1981) pp 165-241 In: Laser Induced Chemical Processes, edited by J. J. Steinfeld; Bi et al. (1993) J. Mater. Res., 8(7): 1666-1674; Bi et al. (1995) J. Mater. Res. 10(11): 2875-2884; Curcio et al. (1990) Applied Surface Science, 46: 225-229; Danen et al. (1984) SPIE, 458: 124-130; Gupta et al. (1984) SPIE, 458: 131-139; U.S. Pat. Nos. 5,958,348, 6,225,007, 6,200,674, 6,080,337, and the like).

Similarly, the most common methods of TiN thin film creation are physical vapor deposition (PVD, usually sputter deposition, Cathodic Arc Deposition or electron beam heating) and chemical vapor deposition (CVD). In both methods, pure titanium is sublimated and reacted with nitrogen in a high-energy, vacuum environment.

Bulk ceramic objects can be fabricated by packing powdered metallic titanium into the desired shape, compressing it to the proper density, then igniting it in an atmosphere of pure nitrogen. The heat released by the chemical reaction between the metal and gas is sufficient to sinter the nitride reaction product into a hard, finished item.

The particles or nanoparticles can be attached to the microcapillaries or cell transfection substrate by any of a number of methods known to those of skill in the art. The particles can simply be sputtered in place, formed on the surface during the formation of metal colloids, grown in place on nucleating particles, ionically attached to the surface, or covalently coupled to the surface, e.g., directly or through a linker/functionalizing agent (e.g., —SH, silane (e.g., 3-aminopropyltirmethoxysilane, and the like), and so forth.

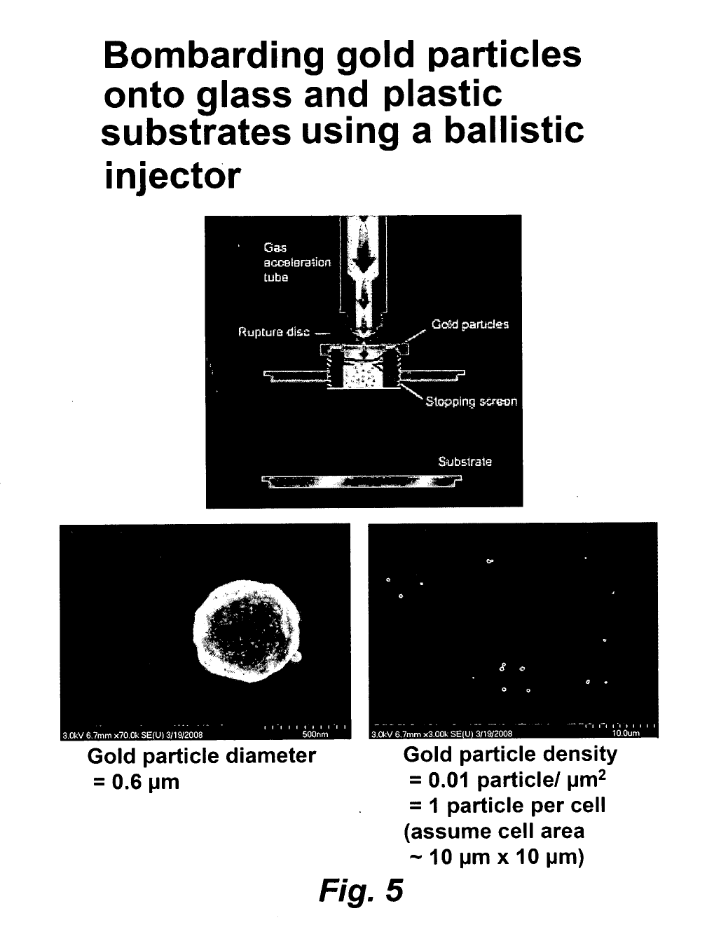

As illustrated in the examples, in one embodiment, the cell transfection substrate is fabricated by bombarding gold particles directly onto a plastic substrate using a biolistic cell injector (BioRad) (see, e.g., Example 4, and FIG. 5).

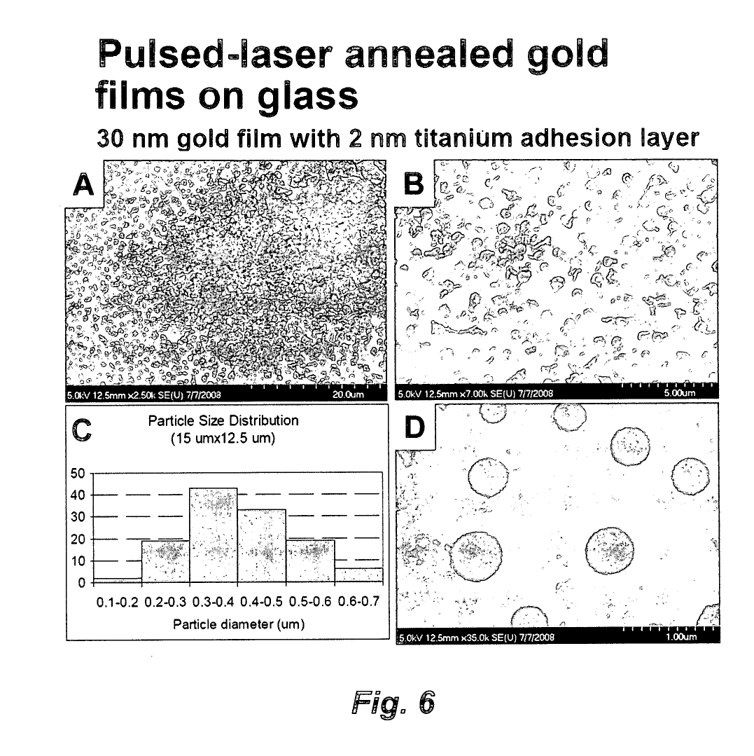

In another approach to it was discovered that heating a thin film on a substrate or micropipette, e.g., using a pulsed laser, can anneal the thin film into disperse nanoparticles. Thus, as illustrated in FIG. 6 fabrication can involve depositing a layer of adhesion metal (e.g. titanium, chromium) followed by a layer of gold (or other metal) film on glass, or plastic, or quartz, etc. substrates. The sample is then heated (e.g., irradiated with laser pulses). At high enough laser energies, the metal films melt, and the molten metal condenses to form beads-like particles (as seen in SEM images in FIG. 6) on the substrate or micropipette.

The optional adhesion metal (e.g., titanium) layer sandwiched between the substrate and the gold layer provides a stronger adhesion of the gold film as well as the gold beads after annealing to the substrate. In the absence of the adhesion layer, gold can still be annealed into nanoparticle beads on the surfaces by laser pulses or other heating methods.

In another approach a substrate or micropipette is provided bearing a thin film. The thin film is then etch away to leave nanoscale size domains that can be heated by applying a laser or other energy source.

The particle array fabrication method(s) can be extended to other micro or nanofabrication techniques such as nanoimprint, e-beam lithography, and others. The current plastic substrate can be replaced with other polymer materials such PDMS or a glass substrate, or a silicon substrate, or others.

The methods of making and attaching particles and nanoparticles to the surface or forming thin films on a surface described above are illustrative and not intended to be limiting. Using the teachings provided herein, other particles, nanoparticles, and thin film coated surfaces can be produced using at most routine experimentation.

Integration with Optical Tweezers.

Conventional microinjection in non-adherent cells is a laborious process since it requires using another holding pipette to apply suction and stabilize the cell. This way the cell has an anchorage to counteract the force exerted by the injection pipette as the pipette penetrates the cell membrane. The suction pressure, relative positioning of the injection pipette and holding pipette can attribute to severe cell damage and death to fragile cells. One current way to increase the microinjection efficiency of non-adherent cells is to bond them on a substrate with treated surface before injection and later on release them from the substrate. This method not only introduces extra chemical treatments to cells and is also time consuming. Optical tweezers have been shown to trap and manipulate micron- and even submicron-sized objects and biological contents. The trapping force of optical tweezers is typically on the order of piconewtons. As a result, it is generally not possible to use optical tweezers to anchor the non-adherent cells during conventional glass microcapillary injections.

The plasmonic photothermal pipette (single surgery tool of this invention), on the other hand, can readily be combined with optical tweezers. In this case, the manipulated cells are self-aligned to the injection position by the optical forces. During the injection process, cells experience minimal sheer force and mechanical distortion which are two critical parameters to keep the injected cells alive. The optical tweezers integrated laser cell surgery technique has the potential to achieve high speed and high efficiency microinjection for non-adherent cells.

### Cell Types

Generally the methods and devices described herein can be used with essentially any cell having a cell membrane. In addition, the methods and devices can also be used on cells having a cell wall.

Thus, for example, adherent cells including NIH3T3 mouse fibroblasts, HEK293T embryonic kidney fibroblasts, and HeLa cervical carcinoma cells have been injected GFP-expressing plasmids using the devices and methods described herein. In general, it is believed that any adherent mammalian cell type can be easily injected using the devices and methods described herein because: 1) the laser fluence that is determined as optimal in terms of effective hole-punching and maintaining cell viability is lies with in a relatively narrow range for all the cell types tested; and 2) adherent cell features used to determine appropriate injection location (e.g., perinuclear or possibly nuclear) are easily identified visually.

Lymphocytes, stem cells of various types, germ cells and others are non-adherent, but it is often desirable to inject or perform other “surgical” procedures on such cells. Integration of optical tweezers with the cell surgery tool as described herein, makes this possible.

In addition, using the methods and devices described herein, injecting individual cells within a cell cluster, such as is required to grow human embryonic stem cells and maintain pluripotency, is achievable especially on the surface of stem cell clusters usng the methods and devices described herein. It is also believed to be possible to stereotactically inject specific cells within clusters, which is desirable for a variety of reasons (e.g, developmental tracking, establishing gradients, etc.).

### Deliverable Materials.

It is believed possible to deliver essentially any desicred material into a cell using the methods and devices described herein. Such materials include, but are not limited to nucleic acids, proteins, organelles, drug delivery nanoparticles, probes, labels, and the like. Delivery of plasmid DNAs into cells using the methods described herein as been demonstrated already in at least three adherent cell types. Accordngly any plasmid-sized genetic material should be easily transferred by the methods and devices described herein.

BACs (bacterial artificial chromosomes)—a desired goal for hard to transduce cells and for delivery vehicles with size restrictions (plasmids, retroviruses, lentiviruses) for introducing large genetic anomalies or for tracking the regulated expression of specific genes during development.

Accordingly it is believed the devices and methods described herein can be used to deliver whole or partial natural or synthetic chromosomes. Similar to BACs, large chromosomes or chromosomal fragments that cannot be transduced into most cell types by previous methods could be transferred into cells by our methods, for example, to establish models of human trisomy disorders (e.g., Down and Klinefelter syndromes).

Similarly the methods can be used for the transfer of nuclei (e.g., in somatic nuclear transfer), or other organelles (e.g., mitochondria, or nanoengineered structures) can readily be introduced into cells.

### Cell Surgery Systems and Patterned Transfection Systems.

In various embodiments this invention contemplates systems for cell surgery or patterned transfection of cells. In certain embodiments the cell surgery systems comprise a microsurgery tool as described herein and a micromanipulator and/or positioner to precisely position the tool, e.g., with respect to a cell. The systems can, optionally, further comprise means for holding cells (e.g., pipettes or other manipulators), means for delivering fluids and/or gases or devices into a cell through the surgical tool, means for removing fluids, organelles, etc., from the cell through the tool, and the like. In certain embodiments the systems can further comprise a viewing system (e.g., a microscope), data/image acquisition systems, and computer control systems for controlling the viewing system, micromanipulators, data/image acquisition systems, and the like.

Similarly, in various embodiments patterned transfection systems comprise a cell transfection substrate (e.g., a culture vessel comprising one or more surfaces bearing particles, nanoparticles, and/or thin films as described herein). The substrate typically bears cells and/or a cell culture. The system can optionally comprise means for delivering reagents, agents to be transfected into the cell(s), means for masking portions of the substrate from an electromagnetic energy source, and the like.

In certain embodiments the systems optionally further include a source of electromagnetic energy to heat the particles, nanoparticles and/or thin film on the surgical tool or transfection substrate. Suitable sources include, but are not limited to a laser, a magnetic field generator, an RF field generator, and the like.

### Kits.

In another embodiment, this invention provides kits for performing single-cell surgery or patterned delivery of an agent into a cell (patterned transfection). In certain embodiments the kits comprise a container containing a single cell surgery tool and/or a transfection substrate as described herein. In various embodiments the kits can optionally additionally include any of the reagents or devices described herein (e.g., reagents, buffers, tubing, indicators, manipulators, etc.) to perform single surgery and/or patterned transfection of cell(s).

In addition, the kits optionally include labeling and/or instructional materials providing directions (i.e., protocols) for the use the surgery tool and/or patterned transfection substrate (e.g., practice of the methods) of this invention. In certain embodiments the instructional materials describe the use of the cell surgery tools described herein to inject or remove materials, and/or to manipulate components of a cell and/or the use of the transfection substrate to deliver one or more agents into a cell.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Fabrication and Use of a Cell Surgery Tool

This example pertains to a novel cell surgery device that integrates nanoparticle photothermal effects with microcapillary techniques. Proof-of-concept experiment results are presented here. The conventional microcapillary technique is a versatile tool for performing single cell recording and manipulations. However, it introduces enormous stress to the cell as the microcapillary punctures through the cell membrane. As a result, this procedure often results in cell death, particularly on small or mechanically fragile cells.

Current cell surgery methods using laser ablation (Vogel et al. (2005) Appl. Phys. B-Lasers O., 81(8): 1015-1047) eliminate the mechanical stress but they require tightly focused light and precise positioning of the injection micropipette at the laser focal spot. The cell surgery device we described in this example, utilizes photothermal effects of nanoparticles on the tip of a microcapillary pipette. Laser-induced heating of the nanoparticles creates transient holes in the cell membrane as the pipette encounters the cell. Since the heating only occurs at the membrane area in contact with nanoparticles, this device can operate with non- or lightly-focused laser. This way unwanted stress is minimized. Possible chemical effects due to strong laser intensity are avoided to ensure the biology of the manipulated cells under study is unaffected.

FIG. 1 shows a schematic of the cell surgery tool. Gold nanoparticles are coated onto the tip of a micropipette. Noble metal nanoparticles strongly absorb electromagnetic waves with a frequency close to its surface plasmon frequency, usually in the visible and NIR range (Hartland (2006) Annu. Rev. Phys. Chem., 57: 403-430). For example, 30 nm diameter gold nanospheres show a peak at wavelengths around 532 nm in their extinction spectrum. Upon laser pulse excitation, the nanoparticles rapidly heat up due to the absorbed energy, causing superheating and evaporation of the surrounding medium. This direct heating or cavitation force from the collapsing vapor bubbles lead to increase in cell membrane permeability or “holes punching” in the membrane. The damage volume can be controlled by laser pulse fluence and nanoparticle' size. It has been shown that the heated volume extends tens of nanometers from the surface of a 30 nm gold nanosphere, and the nanoparticle cools down to equilibrium temperature within few nanoseconds after laser pulsing (Pitsillides et al. (2003) Biophys. J., 84: 4023-4032, 2003; Kotaidis et al. (2006) J. Chem. Phys., 124(18), Art. No. 184702). As a result, the rest of the membrane or cell does not have sufficient time to respond and remains mechanically undisturbed. This way a micropipette can penetrate the cell membrane with ease and cell damage is minimized.

### Experiment and Results.

Synthesis of Gold Nanorods

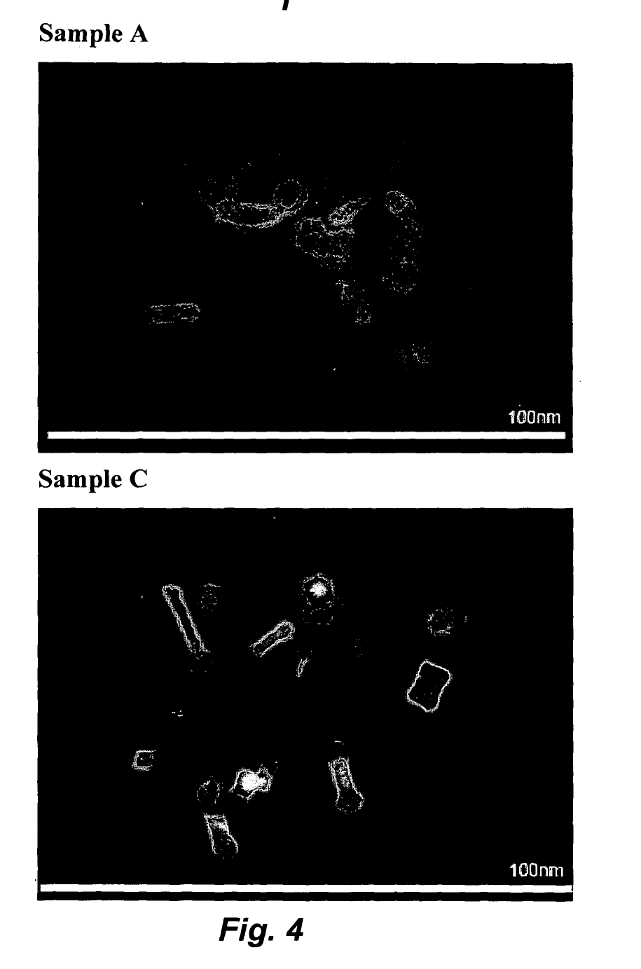

In various embodiments the synthesis of nanorods can be achieved either through the use of rigid templates or surfactants. For our application, we employed the surfactant route for the relatively facile synthetic methods. Briefly, the nanorods synthesized were created via seed mediated growth, developed by Jana et al. (2001) J. Phys. Chem. B, 105(19): 4065-4067, where a 3-4 nm seed added to a growth solution and aged, in the presence of surfactants, to yield nanorods with unique aspect ratios. In addition to surfactant concentration, the aspect ratios have been shown to be controlled by the addition of specific amounts of silver ion (Nikoobakht and El-Sayed (2003) Chem. Mater. 15(10): 1957-1962). The resulting nanorod solutions were characterized via TEM on carbon-coated copper grids. FIG. 4 shows the synthesized nanorods with two aspect ratios of 3.2 and 5.8.

Synthesis Process

Benzyldimehtylammoniumchloride hydrate (BDAC), hexadecyltrimethylammonium bromide (CTAB), L-ascorbic acid, silver nitrate (AgNO3) sodium borohydride (NaBH4), hydrogen tetrachloroaurate (HAuCl4-3H2O) were obtained from Sigma Aldrich. Deionized water (18 M) was used throughout the experiments.

Seed Solution:

5.0 ml of 0.20 M CTAB was mixed with 5.0 ml of 0.0005 M HAuCl₄ with stirring at 25° C. 0.60 ml of ice-cold 0.01 M NaBH₄ was added to the mixture. The resulting solution turned brownish-yellow, from the reduction of gold. The resulting solution was stirred vigorously for two minutes to ensure all the Au had been reduced to Au⁰.

Growth Solution:

20.0 ml of 0.15 M BDAC, and 0.40 g CTAB were combined and sonicated for 20 min at 40° C., to dissolve the CTAB completely. To four separate vials, 200 μl of 0.004 M AgNO₃ was dispensed followed by 5 ml BDAC/CTAB surfactant solution. This produced four different growth solutions, to be aged variably. The growth solution exhibited an orange tinge. To each vial, 5.0 ml of 0.0010 M HAuCl4 was added and gently mixed, followed by 70 μl of 0.0778 M L-ascorbic acid. The orange tinge disappeared during the reduction of Au³⁺ to Au⁰. 12 μl of the seed solution was added to each vial. This produced a reddish color, which points to the reduction of at least 60% of the Au. The resulting solutions were aged for 1 hr, 24 hr, 48 hr, and 72 hr.

The seed Au was directly grown on the glass and followed by dipping the pipette in the growth solution to make the Au particles bigger.

### Use of Cell Surgery Tool.

In experiments described here, a pulsed laser with wavelength of 532 nm and pulse duration of 5 nanoseconds was used. The laser delivered a fluence of 883 J/m² onto a non-focused spot of 5×3 mm². Gold nanoparticles were synthesized directly on glass microcapillaries (FIG. 2) using the method reported by Xu et al. (2004) Chem. Mater., 16(11):2259-2266. Nalm-6 cells (human B cell precursor leukemia) cultured in RPMI were used. Highly localized transient openings of the cell membrane were generated by the photothermal effect of nanoparticles on glass micropipettes, with the dimension of a typical opening close to the micropipette tip size, around 2 μm (FIG. 3C). The cell remained viable after the procedure. A control experiment using a glass micropipette of the same size without gold nanoparticles was also performed. The cell membrane restored its shape instantaneously and showed no sign of hole opening after laser pulsing. We also investigated the laser-induced photothermal effect of an electrically and thermally conductive amorphous carbon coating. A thin film of amorphous carbon was sputtered onto a glass micropipette. Experimental results showed explosive effects extending over a large volume that lysed and killed the cell instantly under the same laser influence (FIG. 3B).

### Conclusion

This example shows a novel cell surgery tool utilizing photothermal effects of metal nanoparticles and provides a proof-of-concept experiment. Localized and transient hole opening on the cell membrane is accomplished using a gold-nanoparticles-coated micropipette compared to instantaneous cell lysing and killing by amorphous carbon coated micropipette.

### Example 2

### Plasmid DNA Transfection and Expression

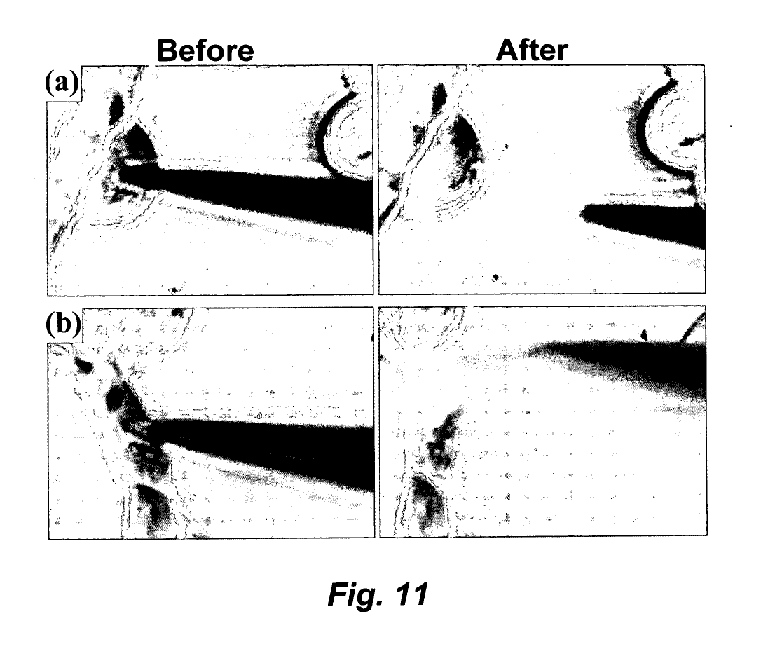

We have demonstrated hole-opening on cell membranes using our plasmonic photothermal pipettes. In the experiments described here, a Q-switched, frequency-doubled Nd:YAG pulsed laser with wavelength of 532 nm and a pulse duration of 6 ns was used (Continuum Minilite I). The laser delivered a fluence of 88.3 mJ/cm² onto a non-focused spot of 11.8 mm². Gold/palladium thin films were deposited onto the glass micropipettes via sputtering. Human embryonic kidney HEK293T cells cultured in DMEM were used. Disruption of the cell membrane was generated by the photothermal effect of the Au/Pd thin film on glass micropipettes. With a film thickness of 5 nm, the dimension of the membrane opening was close to the micropipette tip size, around 2 nm (see, e.g., FIG. 11, panel a). For a film with 13 nm thickness, explosive effects extending over a large volume lysed and killed the cell instantly under the same laser fluence (FIG. 11, panel b). A control experiment of using a glass micropipette of the same size without coating was also performed. The cell membrane showed no sign of hole opening or damage after laser pulsing.

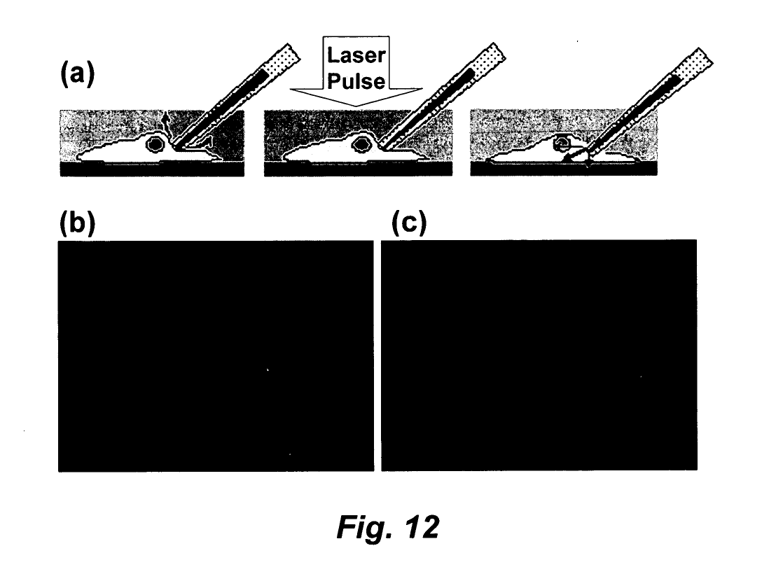

We have also demonstrated microinjection of green fluorescence protein (GFP) encoding plasmids into HEK293T cells. The plasmid is a circular strand of DNA, which upon injection into the cell would allow the cell to produce GFP and fluoresce green. FIG. 12, panel a, shows the microinjection procedure. A continuous flow of plasmids-containing buffer was ejected out of the pipette tip as the photothermal pipette came to a gentle contact with the cell membrane. After applying the laser pulse, the buffer intercalated throughout the cell and the pipette was immediately moved away from the cell. The injected cells were viable 24 hours after the injection and expressed GFP. A separate control experiment was conducted where the pipette ejected out a stream of buffer containing plasmids while in contact with the cell membrane without applying the laser pulse. No cells expressing GFP were found 24 hours later. This is a direct proof that the plasmonic photothermal pipette successfully opened a hole on the cell membrane, which allowed plasmids to flow in. Also the cell survived the procedure and remained viable 24 hours later.

### Example 3

### Integration with Optical Tweezers

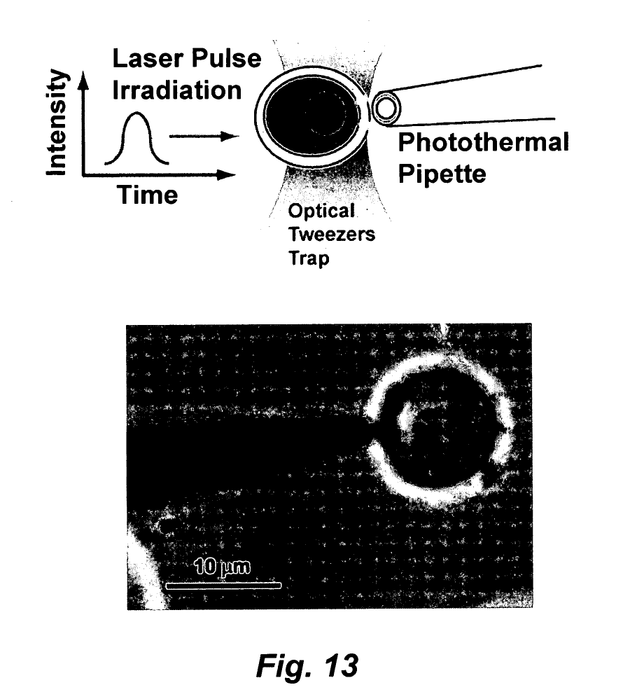

Shown in FIG. 13, a Nalm-6 cell is trapped by a 50 mW, 1064 nm laser beam at the focal point of a N.A. 1.3 100× oil immersed lens while in contact with the photothermal pipette tip. This optical tweezers is constructed on the same Zeiss inverted microscope used for taking time-resolved images of photothermal cell surgery process. This gives us an integrated optical system capable of performing optical trapping, cell surgery, and time-resolved imaging simultaneously. Since the photothermal pipette relies on the nano-bubble explosion to open a hole on the plasma membrane, the pipette tip only needs to be in gentle contact with the cell. In this case optical tweezers provide sufficient trapping force to hold the cell in place during the procedure. Besides minimizing the contact force applied on the cell from both the holding and injection pipettes, another advantage of incorporating optical tweezers is the ease of selecting, trapping and releasing the cell during manipulation.

### Example 4

### Light Image Patterned Molecular Delivery into Live Cells Using Gold Particle Coated Substrate

Optoporation, a method for molecular and gene delivery into cells, utilizes a tightly-focused, pulsed laser beam to create pores in the cell membrane (Vogel et al. (2005) Appl. Phys. B-Lasers O., 81(8): 1015-1047). It allows for contact-free delivery, and with the use of a femtosecond laser, 100% transfection efficiency targeted at single cells has been demonstrated (Tirlapur and Konig (2002) Nature 418: 290-291). One drawback of this approach is that in order to obtain site-specific or patterned cell transfection, the laser beam must scan through every cell, which would be time consuming when large-scale, patterned cell transfection is desired, such as in complex tissues.

Another contact-free method of increasing cell membrane permeability is to use light-absorbing micro- or nanoparticles (Pitsillides et al. (2003) Biophys. J. 84: 4023-4032). Upon irradiation by a short pulse laser, the particles create transient and localized explosive bubbles, that disrupt part of cell membrane adjacent to these particles and leaves the remaining cell structure intact. By controlling the particle size, density and the laser fluence, cell permeablization and transfection can be achieved with high efficiency (Yao et al. (2005) J. Biomed. Optics, 10(6): 064012).

Here we describe a simple device that can spatially select and target cells for molecular delivery by light image patterning. Our approach has the potential of achieving large-scale, image-based molecular and gene delivery of defined pattern into specific cells within complex monolayer mixtures.

### Principle and Device Structure

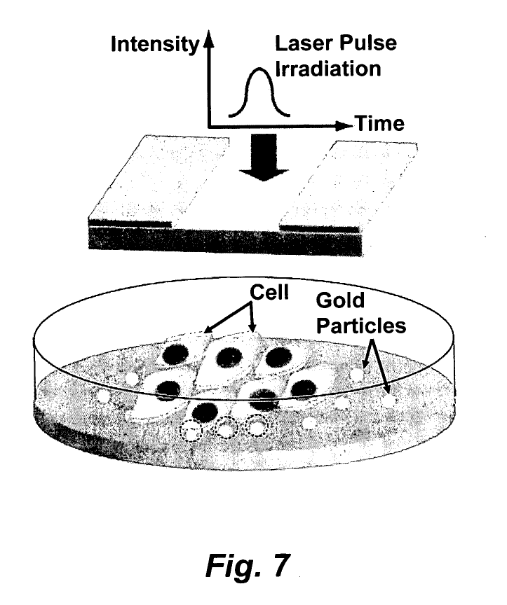

In certain embodiments the device consists of a plastic substrate with particles (e.g., gold particles) immobilized on the surface (see, e.g., FIG. 7). Cells are seeded on this substrate, e.g., until a confluent culture forms. A pulsed laser irradiates a shadow mask and the corresponding illumination pattern is imaged onto the substrate. In the area exposed to the pulsed laser, gold particles are heated to high temperatures due to the absorbed optical energy. Within a few nanoseconds, the heat is dissipated to the thin liquid medium layer surrounding the gold particles, which generates explosive vapor bubbles (Kotaidis et al. (2006) J. Chem. Phys., 124: 184702). The rapid expansion and subsequent collapse of the vapor bubbles give rise to transient fluid flows that induce strong shear stress on the adherent cell causing localized pore formation in the cell membrane. As a result, membrane-impermeable molecules can be carried into the cell by fluid flows or thermal diffusion. Since the cavitation bubble only takes place where the gold particle is exposed to the laser, an optical pattern can be designed to address molecular uptake in specified areas of the cell culture. This way high-throughput, spatially-targeted molecular delivery is made possible by controlling the gold particle size, density on the substrate, and the excitation laser fluence.

### Experiments and Results

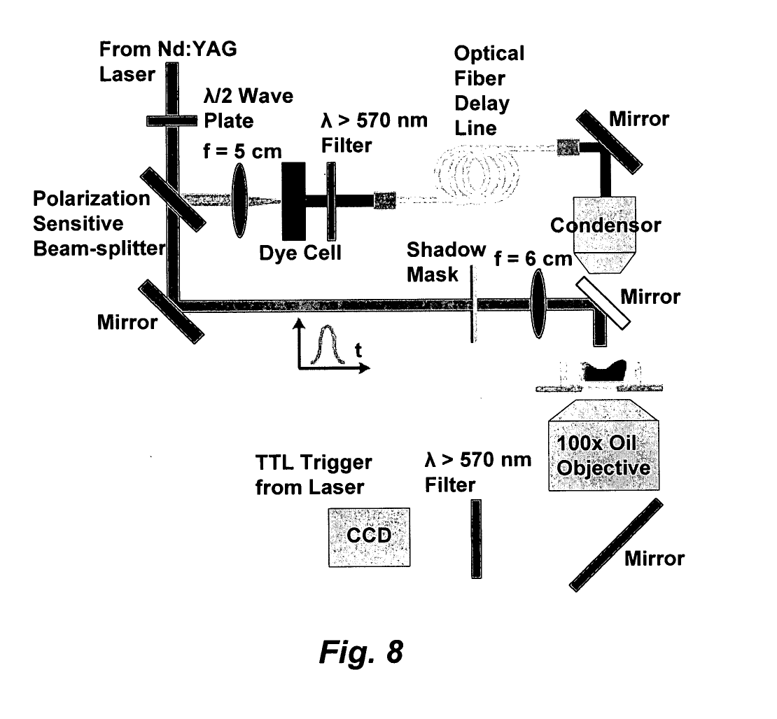

In one experiment, 0.6 μM gold nanospheres (Bio-Rad) were bombarded onto a plastic petri dish using a biolistic injector at 2200 psi bombardment pressure (Bio-Rad, PDS-1000). Immortalized human embryonic kidney cells, (HEK293T) cultured in DMEM were then plated in the dish and incubated overnight until about 70-80% cell confluence was reached. A Q-switched, frequency-doubled Nd:YAG laser at 532 nm in wavelength (Continuum, Minilite I) was used to irradiate the device. The laser has a pulsewidth of 6 nanoseconds and a spot size of 9.4 mm². A shadow mask was placed in the beam path to cast the desired optical pattern, which was imaged onto the device at 0.83× reduction. The induced cavitation bubbles from the gold particles were captured using the time-resolved imaging system depicted in FIG. 8. A high-speed Intensified CCD camera (Princeton Instrument, PI-MAXII) provided exposure times as short as 500 ps. A nanosecond time delay between the captured bubble image and the excitation laser pulse was controlled by the length of an optical fiber delay line. During laser pulsing, cells were immersed in a medium containing the membrane-impermeable fluorescent dye Calcein (Invitrogen, mol wt 622.5) at 1 mg/ml. After cavitation induction, the cell culture was washed with phosphate buffered saline and re-immersed in fresh medium before checking fluorescence staining.

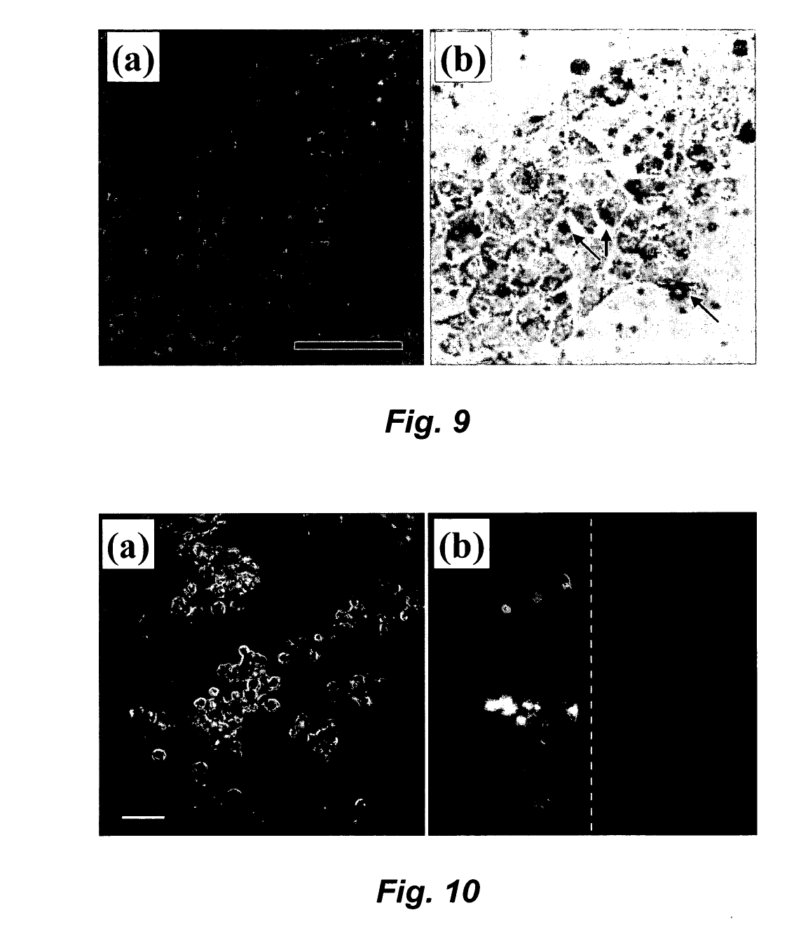

FIG. 9 shows the cavitation bubbles induced by heated gold particles 78 nanoseconds after the laser pulse without a shadow mask. The density of the particles is about 0.004 particles/μm². This corresponds to about 0.9 bubbles per cell (assuming the area of a HEK293T cell is ˜15×15 μm). In FIG. 10, a shadow mask was used and only the left half of the device was irradiated with the laser pulse (dashed line corresponds to the shadow mask boundary). Laser fluence was 128.2 mJ/cm² and 7 pulses were applied. The fluorescent image clearly shows that the dye uptake pattern coincides with the lighted area.

### Conclusion

A device capable of light-patterned molecular delivery is described here.

Successful delivery of fluorescent molecules was demonstrated in adherent cell culture. The targeted delivery area was controlled by a shadow mask. This device has the potential to achieve large-scale, light-patterned molecular and gene delivery in living cells.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A cell microsurgery tool, said tool comprising a microcapillary having at and/or near the tip a thin film coating and/or a plurality of nanoparticles that can be heated by application of electromagnetic energy.

2. The microsurgery tool of claim 1, wherein said thin film coating and/or nanoparticles are comprised of a metal.

3. (canceled)

4. The microsurgery tool of claim 1, wherein the tip of said microcapillary ranges in diameter from about 0.01 μm to about 10 μm.

5. The microsurgery tool of claim 1, wherein the nanoparticles range in size from about 5 nm to about 500 nm.

6. (canceled)

7. The microsurgery tool of claim 1, wherein the nanoparticles are selected from the group consisting of a nanobead, nanowire, a nanotube, a nanodot, a nanocone, and a quantum dot.

8. The microsurgery tool of claim 1, wherein the thin film coating or nanoparticles comprise a material selected from the group consisting of carbon, a metal, a metal alloy, a metal nitride, and a metal oxide.

9. The microsurgery tool of claim 8, wherein the thin film coating or nanoparticles comprise a material selected from the group consisting of a noble metal, a noble metal alloy, a noble metal nitride, a noble metal oxide, a transition metal, a transition metal alloy, a transition metal nitride, a transition metal oxide, a magnetic material, paramagnetic material, and a superparamagnetic material.

10. (canceled)

11. The microsurgery tool of claim 8, wherein the thin film coating or nanoparticles comprise a material selected from the group consisting of gold, titanium (Ti), TiN, TiCn, and TiAlN.

12. (canceled)

13. The microsurgery tool of claim 1, wherein the microcapillary comprises a material selected from the group consisting of glass, a mineral, a ceramic, and a plastic.

14. The microsurgery tool of claim 1, wherein the microcapillary comprises a glass microcapillary having nanoparticles near the tip.

15. The microsurgery tool of claim 1, wherein the microcapillary comprises a glass microcapillary having a thin film comprising titanium near the tip.

16. (canceled)

17. A method of performing micromanipulations on a cell, said method comprising:
contacting said cell with a microsurgery tool, said tool comprising a microcapillary having at and/or near the tip a metal coating or metal nanoparticles that can be heated by application of electromagnetic energy;
applying electromagnetic energy to said tool whereby the temperature of said metal coating or metal nanoparticles is increased thereby facilitating penetration of said tool into or through the membrane of said cell.

18. The method of claim 17, wherein said applying electromagnetic energy comprises applying light to heat the metal coating or the nanoparticles.

19. The method of claim 17, wherein said applying electromagnetic energy comprises applying a laser beam to heat the metal coating or the nanoparticles.

20. The method of claim 17, wherein said applying electromagnetic energy comprises applying a magnetic field or an electric field to heat the metal coating or the nanoparticles.

21. (canceled)

22. The method of claim 17, wherein the temperature of the metal coating or metal nanoparticles is increased at least 150° C. above-ambient.

23-24. (canceled)

25. The method of claim 17, wherein said method further comprises injecting or removing a material into the cell through said microcapillary tube.

26-27. (canceled)

28. The method of claim 17, wherein the tip of said microcapillary ranges in diameter from about 0.01 μm to about 10 μm

29. The method of claim 17, wherein the nanoparticles range in size from about 5 nm to about 500 nm.

30. (canceled)

31. The method of claim 17, wherein the nanoparticles are selected from the group consisting of a nanobead, nanowire, a nanotube, a nanodot, a nanocone, and a quantum dot.

32. The method of claim 17, wherein the metal coating or nanoparticles comprise one or more materials selected from the group consisting of a noble metal, a noble metal alloy, a noble metal nitride, a noble metal oxide, a transition metal, a transition metal alloy, a transition metal nitride, a transition metal oxide, a magnetic material, a paramagnetic material, and asuperparamagnetic material.

33-34. (canceled)

35. The method of claim 17, wherein the microcapillary comprises a material selected from the group consisting of glass, a mineral, a ceramic, and a plastic.

36. The method of claim 17, wherein the microcapillary comprises a glass microcapillary having a thin film metal coating near the tip.

37-39. (canceled)

40. A system for performing microsurgery on a cell, said system comprising a microsurgery tool according to claim 1, and a micromanipulator for positioning said microsurgery tool.

41. The system according to claim 40, further comprising an optical tweezers.

42. The system according to claim 40, further comprising a microscope for visualizing a cell manipulated by said microsurgery tool.

43. The system according to claim 40, further comprising a pump for delivering or removing a molecule, organelle, using said microsurgery tool.

44. The system according to claim 40, further comprising an electromagnetic energy source for exciting the metal nanoparticles and/or thin film on said microsurgery tool.

45. The system according to claim 44, wherein said electromagnetic energy source is selected from the group consisting of a magnetic field generator, a laser, and an RF field generator.

46. A method of preparing a tool for microsurgery on a cell, said method comprising:
attaching to a microcapillary tube a plurality of nanoparticles at or near the tip of said microcapillary tube thereby providing a device that can be locally heated by application of electromagnetic energy to the nanoparticles.

47. The method of claim 46, wherein said attaching comprises adsorbing the nanoparticles to the microcapillary.

48. The method of claim 46, wherein said attaching comprises fabricating the nanoparticles in situ on the microcapillary.

49. The method of claim 46, wherein said attaching comprises chemically coupling the nanoparticles to said microcapillary.

50. A method of delivering an agent into a cell, said method comprising:
providing cells on, or adjacent to, a substrate, where said substrate comprises a metallic thin film and/or metal particles;
contacting said cells with said agent; and
exposing a region of said substrate with electromagnetic radiation thereby inducing heating of said thin film and/or particles where said heating forms bubbles that introduce openings in the membrane of cells in the heated region resulting in the delivery of said agent into those cells.

51. The method of claim 50, wherein said exposing comprises exposing a region of said substrate to a laser.

52. The method of claim 50, wherein said surface comprises a material selected from the group consisting of a glass, a mineral, and a plastic.

53. The method of claim 50, wherein said substrate comprises nanoparticles and said nanoparticles range in size from about 5 nm to about 500 nm.

54. (canceled)

55. The method of claim 53, wherein the nanoparticles are selected from the group consisting of a nanobead, a nanowire, a nanotube, a nanodot, a nanocone, and a quantum dot.

56. The method of claim 50, wherein the metal coating or nanoparticles comprise a material selected from the group consisting of a metal, a metal alloy, a metal nitride, and a metal oxide.

57. The method of claim 50, wherein the metal coating or nanoparticles comprise a material selected from the group consisting of a noble metal, a noble metal alloy, a noble metal nitride, a noble metal oxide, a transition metal, a transition metal alloy, a transition metal nitride, a transition metal oxide, a magnetic material, a paramagnetic material, and a superparamagnetic material.

58. (canceled)

59. The method of claim 50, wherein the metal coating or nanoparticles comprise a material selected from the group consisting of gold, titanium (Ti), TiN, TiCn, and TiAlN.

60. (canceled)

61. The method of claim 50, wherein the substrate comprises a material selected from the group consisting of glass, a mineral, a ceramic, and a plastic.

62. The method of claim 50, wherein said substrate comprises a wall and/or floor of a well in a microtiter plate, a microscope slide, and a cell culture vessel.

63. The method of claim 50, wherein said agent is selected from the group consisting of a nucleic acid, a chromosome, a protein, a label, an organelle, and a small organic molecule.

64. The method of claim 50, wherein said cells are mammalian cells.

65. A device for delivering an agent into a cell, said device comprising:
a vessel comprising a surface bearing nanoparticles or a thin film of a material that heats up when contacted with electromagnetic radiation.

66. The device of claim 65, further comprising a cell culture on said surface.

67. The device of claim 65, wherein said vessel comprises a cell culture vessel.

68. The device of claim 65, wherein said vessel comprises a microtiter plate.

69. (canceled)

70. A system for selectively delivering an agent into a cell, said system comprising:
a vessel comprising a surface bearing nanoparticles or a thin film of a material that heats up when contacted with electromagnetic radiation;
a source of electromagnetic energy capable of heating said nanoparticles or thin film.

71-72. (canceled)

73. A method of fabricating a substrate bearing a plurality of nanoparticles, said method comprising:
providing a substrate; and
bombarding said substrate with nanoparticles whereby said nanoparticles adhere to said substrate; or
providing a substrate bearing a metal film; and
heating said film whereby said film forms metal nanoparticles adhered to said substrate.

74. The method of claim 73, wherein said substrate comprises a material selected from the group consisting of plastic, glass, and quartz.

75. The method of claim 73, wherein said substrate comprises a micropipette, a wall of a cell culture vessel, or a wall of a microtiter plate.

76-82. (canceled)

83. The method of claim 73, wherein said film comprises titanium.

84. The method of claim 73, wherein said film comprises a first film that forms nanoparticles and a second adhesion film where said first film is a different material than said second film.
